(19) United States
(12) Patent Application Publication
(10) Pub. No.: US 2022/0049866 A1
(43) Pub. Date: Feb. 17, 2022

(54) FRAGRANCE CONTAINER OF A SCENTING DEVICE

(71) Applicant: MAHLE International GmbH, Stuttgart (DE)

(72) Inventors: Thomas FEITH, Pforzheim (DE); Andre BAUER, Wendlingen (DE); Christian SCHEURER, Leinfelden-Echterdingen (DE)

(73) Assignee: MAHLE International GmbH, Stuttgart (DE)

(21) Appl. No.: 17/401,817

(22) Filed: Aug. 13, 2021

(30) Foreign Application Priority Data
Aug. 13, 2020 (DE) 10 2020 210 313.5

(51) Int. Cl. F24F 8/50; B60H 3/00; F24F 8/95

(52) U.S. Cl. CPC F24F 8/50; F24F 8/95; B60H 3/0007

(57) ABSTRACT

A fragrance container of a scenting device, having a wall enclosing an interior space, wherein at least one air inlet opening and at least one air outlet opening are provided in the wall for air to flow through the fragrance container, wherein at least one carrier medium for a fragrance is arranged in the interior space of the fragrance container, wherein the at least one carrier medium has a plurality of free surfaces along which the air can flow through from the air inlet opening to the air outlet opening for the purpose of absorbing the fragrance.

This nonprovisional application claims priority under 35 U.S.C. § 119(a) to German Patent Application No. 10 2020 210 313.5, which was filed in Germany on Aug. 13, 2020, and which is herein incorporated by reference.

### BACKGROUND OF THE INVENTION

### Field of the Invention

The present invention relates to a fragrance container of a scenting device, in particular for scenting a vehicle interior. The invention further relates to a scenting device having a fragrance container.

### Description of the Background Art

Scenting devices are widely known in the state of the art. Thus, for example, passive scenting devices are known which release fragrances into the surrounding air on their own, wherein in the case of the passive scenting devices there is no control for releasing the fragrance.

Scenting devices have also become known in which the releasing of the fragrance to an airflow is controlled. In this regard, it is typically the case that the scenting device releases the scent in a metered manner to an airflow in an air duct, wherein for metering the fragrance intensity the amount of fragrance released is determined and released in a controlled manner.

In such scenting devices, the fragrance is absorbed as a liquid in a fragrance container through which the air which is intended to absorb the fragrance flows. For this purpose, the air enters the fragrance container, flows over the free liquid surface of the fragrance, and then exits the fragrance container again. In so doing, the fragrance evaporates and is carried along by the airflow so that it can be olfactorily perceived in the blown-out air.

It turns out that the use of a liquid fragrance can lead to disadvantageous wetting phenomena of the fragrance in the valve of the fragrance control, which is why scenting can be weakened or even completely prevented if the valve is partially or completely clogged by the fragrance. Sloshing phenomena can also occur when using a liquid fragrance, which can lead to the fragrance container overflowing or leaking.

If, in addition to the liquid fragrance, a sponge is placed on the bottom of the fragrance container, which is supposed to soak up the fragrance, in order to reduce the abovementioned sloshing phenomena, the transfer of the fragrance to the air flowing through the fragrance container, however, is worsened such that the scenting is hardly still perceptible.

### SUMMARY OF THE INVENTION

It is therefore an object of the present invention to provide a fragrance container which has a good fragrance release with little sloshing phenomena. It is also the object to provide a scenting device which has an improved fragrance container in which sloshing phenomena are reduced.

An exemplary embodiment of the invention relates to a fragrance container of a scenting device, having a wall enclosing an interior space, wherein at least one air inlet opening and at least one air outlet opening are provided in the wall for air to flow through the fragrance container, wherein at least one carrier medium for a fragrance is disposed in the interior space of the fragrance container, wherein the at least one carrier medium has a plurality of free surfaces along which the air can flow through from the air inlet to the air outlet for the purpose of absorbing the fragrance. This ensures that the fragrance, which is formed at least substantially liquid or gel-like, etc., is absorbed releasably by the carrier medium and is arranged in a spatially defined manner, so that sloshing phenomena are reduced or lessened and nevertheless a large surface is available to be able to release the fragrance easily to the air flowing through the fragrance container.

It is also advantageous if the at least one carrier medium is formed of a sponge-like material and in each case has at least one free surface, preferably a plurality of free surfaces in each case. As a result, the carrier medium can absorb the fragrance well and, due to the at least one free surface or the plurality of free surfaces, the air on this free surface or on these free surfaces can be released well to the air flowing past for scenting. It is particularly advantageous in this regard if a plurality of free surfaces are available in order to be able to suitably increase the surface for the fragrance release.

It is also expedient if the at least one carrier medium consists of foam material, a fabric material, a fleece material, a thread material, a cord material, a batting material, etc. As a result, the carrier medium can absorb the fragrance well and also release it again when an airflow flows along at least one free surface of the carrier medium.

Furthermore, it is also expedient if the at least one carrier medium is formed plate-shaped, rod-shaped, and/or tubular. As a result, the carrier medium can be easily manufactured as a prefabricated component and can be securely disposed in the fragrance container. At the same time, due to this design the spacing of free surfaces can also be designed for the air to flow through for improved fragrance absorption by the air.

It is also advantageous if a plurality of carrier media are provided and disposed in the interior space, wherein at least two carrier media or the carrier media are arranged at a distance from one another such that at least one path through which air can flow remains between single carrier media or between the carrier media. As a result, multiple air paths can advantageously be designed between carrier media and/or within a carrier medium in order to improve the scenting.

It is also expedient if the wall has an opening that can be closed by a cover for inserting and/or exchanging the at least one carrier medium and/or for filling the fragrance. As a result, the fragrance container can be opened and provided with the carrier medium or with the carrier media, so that it or they can absorb the filled fragrance. The respective carrier medium can preferably also be wetted with the fragrance before the carrier medium is inserted into the fragrance container.

It is also advantageous if the wall has a holder or slotted guides for the defined arrangement and/or guiding of the at least one carrier medium or carrier media. As a result, the carrier medium or the carrier media can be inserted and held in the correct position, so that the free surfaces can be kept free for the inflow of air, so that the air can be scented with no impairment.

It is also expedient if the wall surrounding the interior space has a quadrangular or cylindrical shape. As a result, a simple arrangeable configuration for the position of the air inlet opening and the air outlet opening can be designed.

It is also advantageous if the at least one air inlet opening is arranged substantially opposite the at least one air outlet opening, in particular the at least one air inlet opening being arranged in a first end wall of the wall substantially opposite a second end wall in which the at least one air outlet opening is located. This ensures a good flow through the fragrance container, which promotes the scenting of the air.

An exemplary embodiment of the invention also relates to a scenting device having at least one air duct, which has an air inlet opening and an air outlet opening, wherein a fan is provided which sucks in or blows air through the air inlet opening into the air duct and conveys it to the air outlet opening, wherein a fragrance container for enriching the air flowing through the fragrance container is provided in the air duct. The fragrance container is designed in particular according to the invention.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 is a schematic representation of a scenting device of the invention with a schematically indicated fragrance container of the invention;

FIG. 2 is a representation of an exemplary embodiment of a fragrance container of the invention;

FIG. 3 is a representation of an exemplary embodiment of a fragrance container of the invention; and

FIG. 4 is a representation of an exemplary embodiment of a fragrance container of the invention.

### DETAILED DESCRIPTION

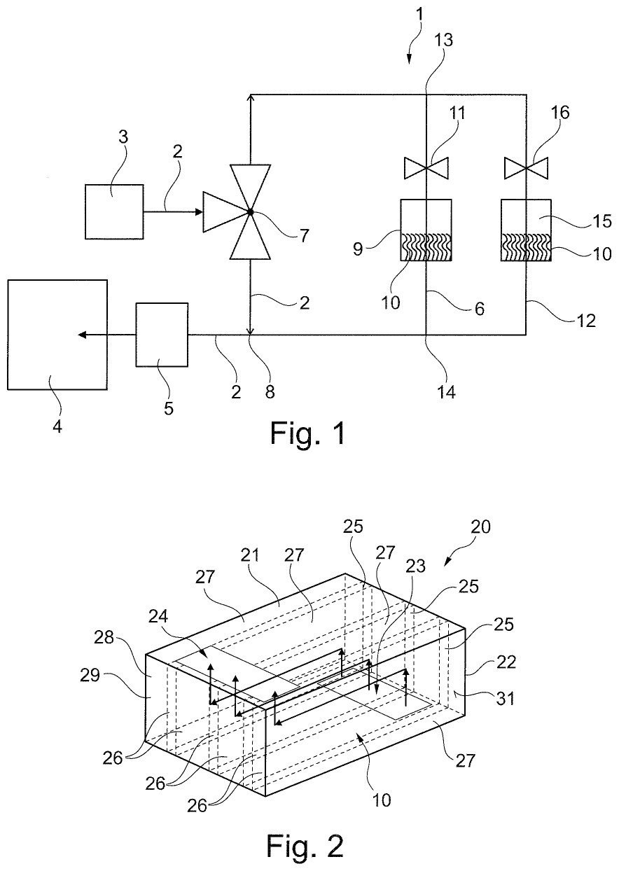

FIG. 1 shows in a schematic representation a scenting device 1 with a scent container 9 of the invention. In this exemplary embodiment shown, a second fragrance container 15 of the invention is optionally also provided in order to use possibly two different fragrances in order to scent the air. Alternatively, further optional fragrance containers can also be provided, but these are not shown.

Scenting device 1 has a first air duct 2 which is connected or formed with an air inlet opening 3 and with an air outlet opening 4. Thus, air can reach first air duct 2 through air inlet opening 3 and the air can be discharged again out of first air duct 2 through air outlet opening 4.

To control the flow, a fan 5 is provided which sucks in or blows air through air inlet opening 3 into first air duct 2 and conveys it to air outlet opening 4. Fan 5 is, for example, a spiral fan.

At least one second air duct 6 is also provided, which branches off from first air duct 2 at a first duct branch 7 and reunites with first air duct 2 at a second duct branch 8. As a result, second air duct 6 is in fluid contact with air inlet opening 3 and with air outlet opening 4, so that air can be directed through air inlet opening 3 to second air duct 6 and from second air duct 6 to air outlet opening 4.

In second air duct 6, a first fragrance container 9 is provided between first duct branch 7 and second duct branch 8, said container through which the airflow in second air duct 6 can flow.

As a result, a quantity of fragrance 10 can be released from fragrance container 9 to the air flowing through fragrance container 9, so that the air absorbs fragrance 10 and carries it away to scent a vehicle interior of a motor vehicle.

To control the flow through fragrance container 9 and thus also the release of fragrance 10 to the air flowing therethrough, a first valve device 11 is provided upstream and/or downstream of first fragrance container 9. In FIG. 1, first valve device 11 is arranged upstream of fragrance container 9. Alternatively or in addition, a valve device (not shown) can also be arranged downstream of fragrance container 9.

An exemplary method for operating scenting device 1 advantageously provides that fan 5 is controlled such that a defined, substantially constant airflow is generated as a total through first air duct 2 and second air duct 6, wherein the degree of scenting is controlled by the controlled amount of fragrance released to the airflow in second air duct 6 and/or by the amount of air in the airflow in second air duct 6. First air duct 2 and second air duct 6 are connected more or less in parallel when valve device 11 is open in order to generate a defined overall airflow.

According to the optional refinement shown of scenting device 1, a third air duct 12 can also be provided, which branches off from first air duct 2 at a third duct branch 13 and reunites with first air duct 2 at a fourth duct branch 14, wherein a second fragrance container 15 is provided in third air duct 12 between third duct branch 13 and fourth duct branch 14, said container through which the airflow in third air duct 12 can flow.

In this regard, third air duct 12 can branch off directly or indirectly from first air duct 2 and/or unite again directly or indirectly with first air duct 2.

It can be the case, for example, that third air duct 12 branches off indirectly from first air duct 2 in that it branches off from second air duct 6 and/or third air duct 12 indirectly reunites with first air duct 2 by merging with second air duct 6; also see FIG. 1.

In this regard, a second valve device 16, which controls the flow through second fragrance container 15, is advantageously provided upstream and/or downstream of second fragrance container 15. In FIG. 1, the optional second valve device 16 is arranged upstream of second fragrance container 15. Alternatively or in addition, a valve device (not shown) can also be arranged downstream of second fragrance container 15.

For example, fan 5 can be controlled such that a defined, in particular substantially constant airflow is generated through first air duct 2 and/or second air duct 6 and/or third air duct 12, wherein the degree of scenting is controlled by the controlled amount of fragrance released to the airflow in second air duct 6 and/or third air duct 12 and/or by the amount of air in the airflow in second air duct 6 and/or in third air duct 12.

In this regard, the airflow enriched with fragrance 10 through second air duct 6 and/or the airflow enriched with a fragrance 10 through third air duct 12 are mixed with the airflow through first air duct 2. The total air flow remains substantially unchanged in this case.

FIG. 2 shows a detailed representation of a fragrance container 20 of the invention of a scenting device 1. Fragrance container 20 has a wall 22 enclosing an interior space 21.

For air inflow, at least one air inlet opening 23 and at least one air outlet opening 24 are provided in wall 22 for air to flow through fragrance container 20.

At least one carrier medium 25 for a fragrance 10 is arranged in interior space 21 of fragrance container 20. In the exemplary embodiment shown, three carrier media 25 are provided, which are formed as essentially plate-shaped carrier media 25 and which are arranged at a distance from one another.

In this regard, the at least one carrier medium 25 has a plurality of free surfaces 26. In the exemplary embodiment shown, the three carrier media 25 have a plurality of free surfaces 26. A free surface 26 is a surface of carrier medium 25 that is exposed to the airflow and along which air can flow to absorb a fragrance 10. In the exemplary embodiment shown, each of the three carrier media 25 has two free surfaces 26 which are each formed by the large side surfaces of the substantially plate-shaped carrier medium 25.

FIG. 2 shows an exemplary embodiment in which multiple carrier media 25 are provided and arranged in interior space 21, wherein at least two carrier media 25 or carrier media 25 are arranged at a distance from one another such that at least one path 27 through which air can flow remains between single carrier media 25 or between carrier media 25. Paths 27 through which air can flow are also provided between the respectively outer carrier medium 25 and wall 22.

The air can flow along free surfaces 26 along paths 27 from the air inlet at air inlet opening 23 of fragrance container 20 to the air outlet at air outlet opening 24 of fragrance container 20 for absorbing the fragrance.

For absorbing and storing fragrance 10 and for again releasing fragrance 10, the at least one carrier medium 25 is made of a sponge-like material and in each case has at least one free surface 26, preferably a plurality of free surfaces 26.

FIG. 2 shows that the at least one carrier medium 25 is formed plate-shaped. Alternatively, carrier medium 25 can also be formed rod-shaped and/or tubular or formed designed in some other way. In principle, carrier media 25 in a fragrance container 20 can be of the same design (see FIG. 2), or different configurations can also be provided in a fragrance container.

Carrier medium 25 can consist, for example, of a foam material, a fabric material, a fleece material, a thread material, a cord material, a batting material, etc.

Fragrance container 20 is advantageously formed such that wall 22 has an opening 29 that can be closed by a cover 28 for inserting and/or exchanging the at least one carrier medium 25 and/or for filling fragrance 10.

For an advantageous design, it is also optional if wall 22 has a holder 31 or slotted guides for the defined arrangement and/or guiding of the at least one carrier medium 25 or carrier media 25.

According to FIG. 2, it can be seen that wall 22 surrounding interior space 21 has a quadrangular design. Alternative designs are also optionally possible, for example, a cylindrical design, a regular or irregular design.

It can be seen further in FIG. 2 that the at least one air inlet opening 23 is arranged substantially opposite the at least one air outlet opening 24.

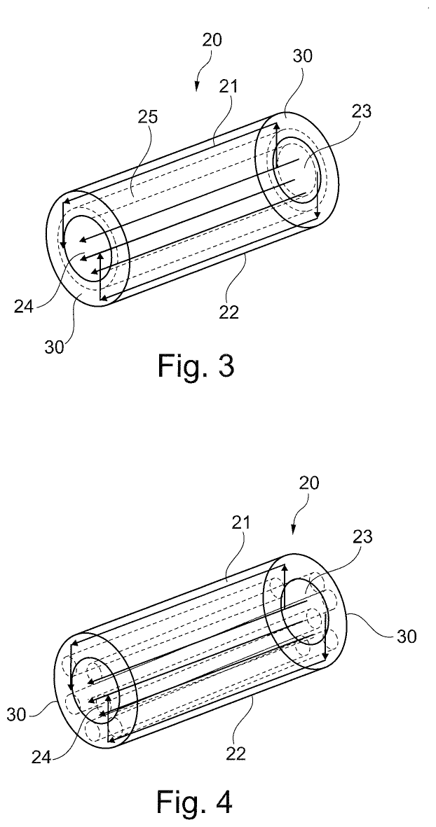

FIGS. 3 and 4 show exemplary embodiments of fragrance containers 20 with a cylindrical design of a wall 22, wherein in FIG. 3 a hollow cylindrical carrier medium 25 is used, which is designed to be flowed around on the outside and flowed through on the inside. In FIG. 4, rod-shaped, cylindrical carrier media 25 are used, which are arranged to be flowed around.

Furthermore, the at least one air inlet opening 23 is arranged in a first end wall 30 of wall 22 substantially opposite a second end wall 30 in which the at least one air outlet opening 24 is located.

Scenting device 1 can be designed as an autonomous device, which is not designed as an integrated component of the motor vehicle but which can be operated and positioned independently. In this regard, there can be an independent power supply via batteries or rechargeable batteries or an electrical supply connection to the vehicle electrical system can be provided.

Alternatively, scenting device 1 could also be part of the vehicle equipment, however.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A fragrance container of a scenting device, the fragrance container comprising:
a wall enclosing an interior space;
at least one air inlet opening and at least one air outlet opening being provided in the wall for air to flow through the fragrance container; and
at least one carrier medium for a fragrance arranged in the interior space of the fragrance container, the at least one carrier medium having a plurality of free surfaces along which air is adapted to flow through from the air inlet opening to the air outlet opening for the purpose of absorbing the fragrance.

2. The fragrance container according to claim 1, wherein the at least one carrier medium is formed of a sponge-like material and has at least one free surface or a plurality of free surfaces.

3. The fragrance container according to claim 1, wherein the at least one carrier medium is formed of a foam material, a fabric material, a fleece material, a thread material, a cord material, or a batting material.

4. The fragrance container according to claim 1, wherein the at least one carrier medium is formed plate-shaped, rod-shaped, and/or tubular.

5. The fragrance container according to claim 1, wherein a plurality of carrier media are provided and disposed in the interior space, wherein at least two carrier media or the carrier media are arranged at a distance from one another such that at least one path through which air is adapted to flow remains between single carrier media or between the carrier media.

6. The fragrance container according to claim 1, wherein the wall has an opening (29) that is adapted to be closed by a cover for inserting and/or exchanging the at least one carrier medium and/or for filling the fragrance.

7. The fragrance container according to claim 1, wherein the wall has a holder or slotted guides for the defined arrangement and/or guiding of the at least one carrier medium or the carrier media.

8. The fragrance container according to claim 1, wherein the wall surrounding the interior space has a quadrangular or cylindrical shape.

9. The fragrance container according to claim 1, wherein the at least one air inlet opening is arranged substantially opposite the at least one air outlet opening or wherein the at least one air inlet opening is arranged in a first end wall of the wall substantially opposite a second end wall in which the at least one air outlet opening is located.

10. A scenting device comprising:
at least one air duct, which has an air inlet opening and an air outlet opening;
a fan that sucks in or blows air through the air inlet opening into the air duct and conveys it to the air outlet opening; and
a fragrance container to enrich the air flowing through the fragrance container being provided in the air duct,
wherein the fragrance container is the fragrance container according to claim 1.
